(19) United States
(12) Patent Application Publication
(10) Pub. No.: US 2003/0204257 A1
(43) Pub. Date: Oct. 30, 2003

(54) Intraocular lens

(76) Inventor: Michael A. Southard, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/134,765

(22) Filed: Apr. 29, 2002

(51) Int. Cl. A61F 2/16

(52) U.S. Cl. 623/6.46; 623/6.49

(57) ABSTRACT

A foldable lens having a reinforced haptic/optic intersection that allows the optic to vault posteriorly unidirectionally while still providing stable fixation of the lens within the eye.

This invention relates to intraocular lenses (IOLs) and more particularly to soft, foldable intraocular lenses.

### BACKGROUND OF THE INVENTION

The human eye in its simplest terms functions to provide vision by transmitting and refracting light through a clear outer portion called the cornea, and further focusing the image by way of lens onto the retina at the back of the eye. The quality of the focused image depends on many factors including the size, shape and length of the eye, and the shape and transparency of the cornea and lens. When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft foldable IOLs made from silicone, soft acrylics and hydrogels have become increasingly popular because of the ability to fold or roll these soft lenses and insert them through a smaller incision. While early foldable lenses either had a plate-style haptic (e.g., U.S. Pat. No. 4,664,666 (Barrett), the entire contents of which being incorporated herein by reference) or were of a multi-piece design with independently formed, relatively rigid haptic attached to the soft optic (e.g., U.S. Pat. No. 5,118,452 (Lindsey, et al.), the entire contents of which being incorporated herein by reference), newer lens designs are of an open-loop variety and manufactured from a single piece (e.g., U.S. Pat. No. 5,716,403, (Tran, et al.), the entire contents of which being incorporated herein by reference). The problem with soft, single-piece, open loop IOLs is that the haptics lack force sufficient to vault the optic posteriorly away from the iris and ensure firm contact with the posterior capsule.

Accordingly, a need continues to exist for a vaulted, single-piece, open loop, soft intraocular lens.

### BRIEF SUMMARY OF THE INVENTION

The present invention improves upon prior art single-piece, open loop, soft intraocular lenses by providing a foldable lens having a reinforced haptic/optic intersection that allows the optic to vault posteriorly unidirectionally while still providing stable fixation of the lens within the eye.

It is accordingly an object of the present invention to provide a stable intraocular lens.

It is a further object of the present invention to provide a vaulted, single-piece, open loop, soft intraocular lens.

It is a further object of the present invention to provide a single-piece, open loop, soft intraocular lens having a stiffened haptic/optic intersection that allows the optic to vault posteriorly.

Other objectives, features and advantages of the present invention will become apparent with reference to the drawings, and the following description of the drawings and claims.

### BRIEF DESCRIPTION OF THE DRAWINGS

FIG. 1 is a top plan view of a first embodiment of an intraocular lens of the present invention.

FIG. 2 is a partial cross-sectional view of the lens illustrated in FIG. 1.

FIG. 3 is a top plan view of a second embodiment of an intraocular lens of the present invention.

FIG. 4 is a partial cross-sectional view of the lens illustrated in FIG. 3.

### DETAILED DESCRIPTION OF THE INVENTION

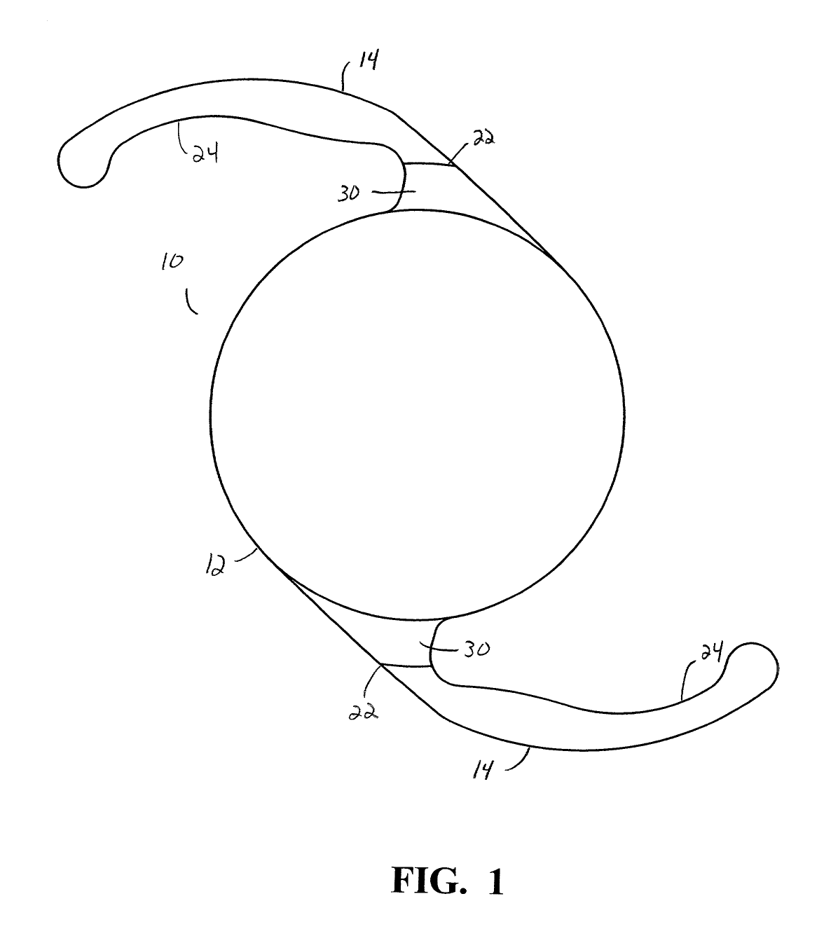

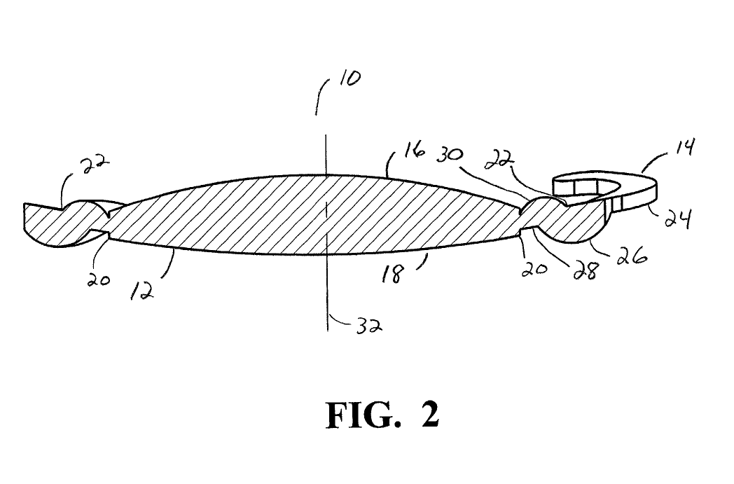

As best seen in FIGS. 1 and 2, in a first embodiment of the present invention, lens 10 generally consists of optic 12 and at least one haptic 14. Optic 12 has an anterior face 16 and a posterior face 18. The overall design of lens 10 may be any suitable single-piece, open loop design made from a soft, foldable material, such as silicone, hydrogel or soft acrylic. One suitable design is illustrated in FIG. 1, and another suitable design is disclosed in U.S. Pat. No. 5,716,403, (Tran, et al.). Haptic 14 intersects edge 20 of optic 12 at an angle relative to the plane of the optic (the plane of the optic being perpendicular to optical axis 32), preferably between 1° and 20°, and most preferably between 5° and 10° but other suitable angles may also be used. Haptic 14 contains joint 22 that acts like a hinge to allow distal section 24 of haptic 14 to pivot anteriorly. Thickened section 26, on the posterior side of haptic 14, helps to assure that flexing of haptic 14 about joint 22 causes distal section 24 of haptic 14 to pivot anteriorly. Proximal portion 28 of haptic 14 contains convex anterior side 30 which assists in transferring compressive forces on haptic 14 into posterior movement of optic 12 along optical axis 32. While any suitable dimensions can be used, haptic 14 preferably is between about 0.10 mm and 0.25 mm thick, with around 0.20 mm being most preferred. Thickened section 26 of haptic 14 preferably is between about 0.40 mm and 0.65 mm thick, with around 0.55 mm being most preferred. Proximal portion 28 of haptic 14 preferably is between about 0.25 mm and 0.55 mm thick, with around 0.40 mm being most preferred. Convex anterior side 30 of proximal portion 28 of haptic 14 preferably is formed with a radius of between about 0.2 mm and 0.6 mm, with around 0.4 mm being most preferred.

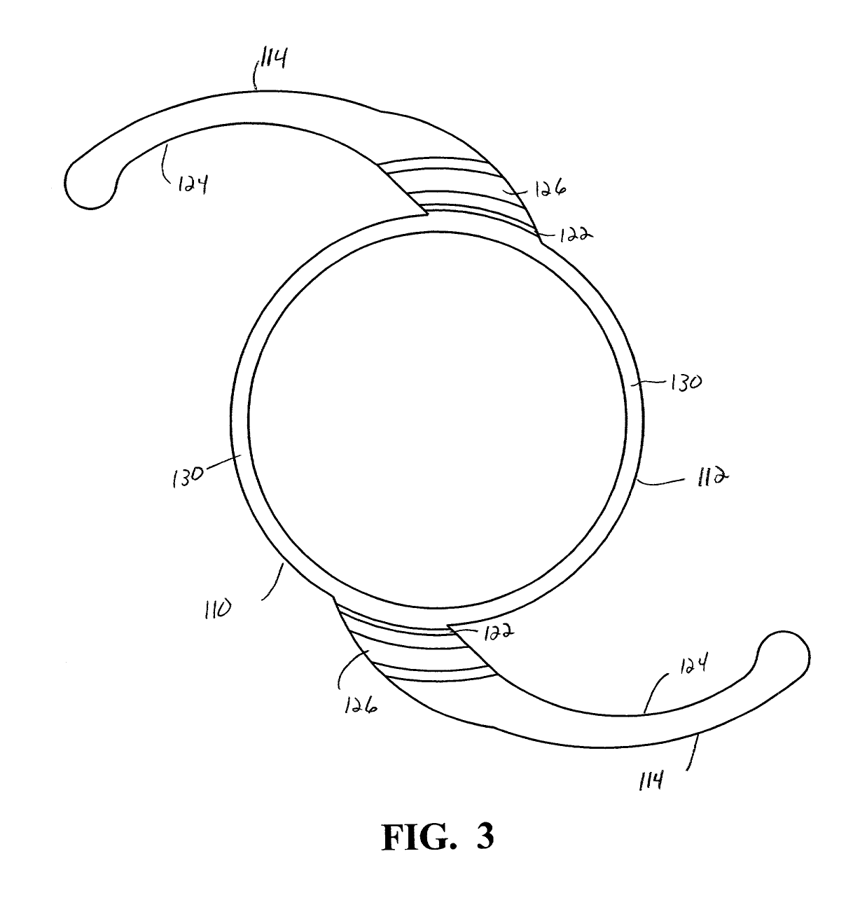

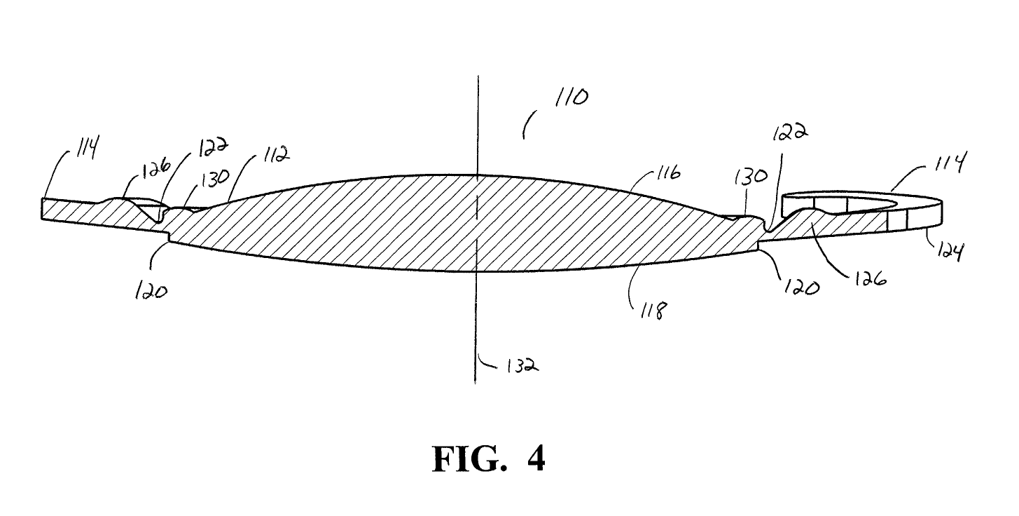

As best seen in FIGS. 3 and 4, in a second embodiment of the present invention, lens 110 generally consists of optic 112 and at least one haptic 114. Optic 112 has an anterior face 116 and a posterior face 118. The overall design of lens 110 may be any suitable single-piece, open loop design made from a soft, foldable material, such as silicone, hydrogel or soft acrylic. One suitable design is illustrated in FIG. 3, and another suitable design is disclosed in U.S. Pat. No. 5,716,403, (Tran, et al.). Haptic 114 intersects edge 120 of optic 112 at an angle relative to the plane of the optic (the plane of the optic being perpendicular to optical axis 132), preferably between 1° and 20°, and most preferably between 5° and 10° but other suitable angles may also be used. Haptic 114 contains joint 122 that acts like a hinge to allow distal section 124 of haptic 114 to pivot anteriorly. Thickened section 126, on the anterior side of haptic 114 and located distally of joint 122, helps to assure that flexing of haptic 114 about joint 122 causes distal section 124 of haptic 114 to pivot anteriorly. Edge 120 of optic 112 contains circumferential stiffening rib 130 at the intersection of joint 122 and edge 120 which assists in transferring compressive forces on haptic 114 into posterior movement of optic 112 along optical axis 132. While any suitable dimensions can be used, haptic 114 preferably is between about 0.10 mm and 0.25 mm thick, with around 0.20 mm being most preferred. Thickened section 126 of haptic 114 preferably is between about 0.20 mm and 0.40 mm thick, with around 0.29 mm being most preferred, with a radius of between about 0.2 mm and 0.4 mm, with around 0.3 mm being most preferred. Joint 122 preferably is between about 0.05 mm and 0.10 mm thick, with around 0.08 mm being most preferred.

While certain embodiments of the present invention have been described above, these descriptions are given for purposes of illustration and explanation. Variations, changes, modifications, and departures from the systems and methods disclosed above may be adopted without departure from the scope or spirit of the present invention.

I claim:

1. An intraocular lens, comprising:
a) an optic made from a foldable material; and
b) at least one open-loop haptic integrally formed with the optic as a single piece, the haptic intersecting the optic at an angle relative to the plane of the optic,
wherein the haptic has a proximal portion at the intersection of the haptic with the optic, the proximal portion being convex on an anterior side and the haptic further containing a posterior side containing a thickened section, the thickened section located distally from the proximal section so as to form a joint between the proximal section and the thickened section, the joint permitting the haptic to flex, thereby moving the optic posteriorly along an optical axis.

2. The intraocular lens of claim 1 wherein the thickened section is between 2; about 0.40 mm and 0.65 mm thick.

3. The intraocular lens of claim 1 wherein the proximal portion is between about 0.25 mm and 0.55 mm thick.

4. An intraocular lens, comprising:
a) an optic made from a foldable material; and
b) at least one open-loop haptic integrally formed with the optic as a single piece, the haptic intersecting the optic at an angle relative to the plane of the optic,
wherein the haptic has a proximal portion at the intersection of the haptic with the optic, the proximal portion being convex on an anterior side and between about 0.25 mm and 0.55 mm thick and the haptic further containing a posterior side containing a thickened section, the thickened section being between about 0.40 mm and 0.65 mm thick and located distally from the proximal section so as to form a joint between the proximal section and the thickened section, the joint permitting the haptic to flex, thereby moving the optic posteriorly along an optical axis.

5. An intraocular lens, comprising:
a) an optic having a circumferential stiffening rib, the optic being made from a foldable material; and
b) at least one open-loop haptic integrally formed with the optic as a single piece, the haptic intersecting the optic at an angle relative to the plane of the optic,
wherein the haptic has a joint at the intersection of the haptic with the optic and a thickened section positioned distally of the joint, the joint permitting the haptic to flex, thereby moving the optic posteriorly along an optical axis.

6. The intraocular lens of claim 5 wherein the thicken section is between about 0.20 mm and 0.40 mm thick.

7. The intraocular lens of claim 5 wherein the joint is between about 0.05 mm and 0.10 mm thick.

8. An intraocular lens, comprising:
a) an optic having a circumferential stiffening rib, the optic being made from a foldable material; and
b) at least one open-loop haptic integrally formed with the optic as a single piece, the haptic intersecting the optic at an angle relative to the plane of the optic,
wherein the haptic has a joint at the intersection of the haptic with the optic the being between about 0.05 mm and 0.10 mm thick and a thickened section being about 0.20 mm and 0.40 mm thick and positioned distally of the joint, the joint permitting the haptic to flex, thereby moving the optic posteriorly along an optical axis.
